(19) United States
(12) Patent Application Publication
(10) Pub. No.: US 2012/0178937 A1
(43) Pub. Date: Jul. 12, 2012

(54) PROCESS FOR THE PREPARATION OF ALOSETRON

(75) Inventors: Buchi Reddy REGURI, Chennai (IN); Sampathkumar UPPARAPALLI, Chennai (IN); Nilam SAHU, Chennai (IN); Karunakara Rao JAVVAJI, Chennai (IN); Brahma Reddy GADE, Chennai (IN)

(73) Assignee: ORCHID CHEMICALS & PHARMACEUTICALS LIMITED, Chennai (IN)

(21) Appl. No.: 13/348,930

(22) Filed: Jan. 12, 2012

(30) Foreign Application Priority Data
Jan. 12, 2011 (IN) 96/CHE/2011

(51) Int. Cl. C07D 471/04

(52) U.S. Cl. 546/86

(57) ABSTRACT

The present invention provides an improved process for the preparation of Alosetron of formula (I) and its pharmaceutically acceptable salts.

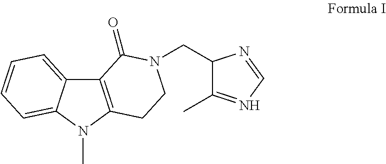

The following specification describes the nature of the invention and manner in which it has to be performed:

### FIELD OF THE INVENTION

The present invention provides an improved process for the preparation of Alosetron of formula (I) and its pharmaceutically acceptable salts.

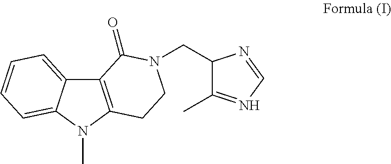

### BACKGROUND OF THE INVENTION

The Alosetron hydrochloride is a potent and selective antagonist of the serotonin 5-HT3 receptor type. Chemically, Alosetron is designated as 2,3,4,5-tetrahydro-5-methyl-2-[(5-methyl-1H-imidazol-4-yl)methyl]-1H-pyrido[4,3-b]indol-1-one, monohydrochloride. This is marketed in United States under trade name of LOTRONEX®

U.S. Pat. No. 5,360,800 discloses a preparation of Alosetron by condensing 2,3,4,5-tetrahydro-5-methyl-1H-pyrido[4,3-b]indol-1-one with 4-chloromethyl-5-methylimidazole in presence of strong base such as sodium hydride. The sodium hydride was corrosive and highly flammable. This type of reaction required extra care, special type of equipments and it is commercially not feasible. This process also provides low yield.

U.S. Pat. No. 6,175,014 patent describes a process for the process Alosetron by reacting of 2,3,4,5-tetrahydro-5-methyl-1H-pyrido[4,3-b]indol-1-one of formula (II) with 4-hydroxymethyl-5-methylimidazole of formula (IIIa) or its salt in presence of mineral acid like hydrochloric acid or sulfonic acids such as p-toluene sulfonic acid or methane sulfonic acid. The process requires purification to get pure Alosetron.

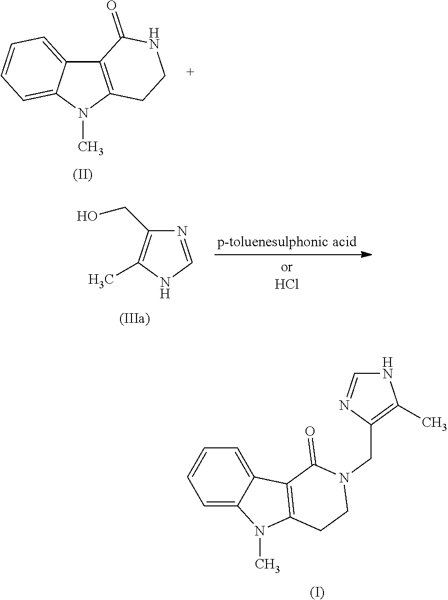

Hence there is a need for a simple and commercially viable process for the preparation of Alosetron which avoids hazardous base such as sodium hydride.

The present inventors identified a new process for the preparation of Alosetron by reaction of 2,3,4,5-tetrahydro-5-methyl-1H-pyrido[4,3-b]indol-1-one of formula (II) with 4-hydroxymethyl-5-methylimidazole of formula (III) or its protected derivative. This process is simple to carryout for large scale preparation and industrially viable. The present inventors also identified a process for the purification of Alosetron to get more pure Alosetron.

### OBJECTIVES OF THE INVENTION

An objective of the present invention is to provide an improved process for the preparation of Alosetron of formula (I) and its pharmaceutically acceptable salts in good yield.

Yet another objective of the present invention is to provide an improved process for the preparation of Alosetron of formula (I) and its pharmaceutically acceptable salts.

Still another objective of the present invention is to provide a purification process for Alosetron of formula (I) and its pharmaceutically acceptable salts.

### SUMMARY OF THE INVENTION

Accordingly, the present invention directed to an improved process for the preparation of Alosetron of formula (I) and its pharmaceutically acceptable salts which comprises
a) condensing 2,3,4,5-tetrahydro-5-methyl-1H-pyrido[4,3-b]indol-1-one of formula (II) with 4-hydroxymethyl-5-methylimidazole of formula (III) in presence of trifluoroacetic acid and in the presence or absence solvent to obtain Alosetron of formula (I); and
b) optionally purifying the Alosetron by using an organic acid.

The present invention is represented by following scheme.

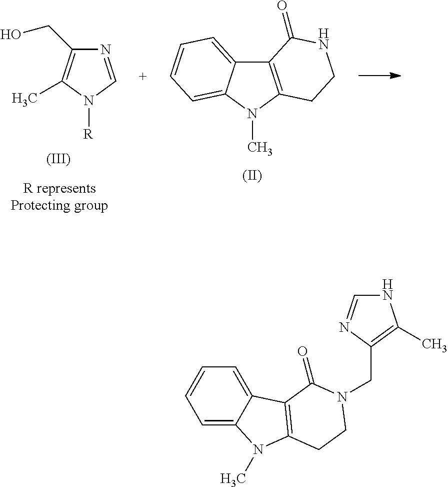

### DETAILED DESCRIPTION OF THE INVENTION

In an embodiment of the present invention, the solvent used for the condensation is selected from dimethyl acetamine, dimethyl formamide, dimethyl sulfoxide, sulfolane, xylene, methanol, ethanol, isopropanol, butanol, acetic acid, formic acid etc.

Applicant found that the use of acetic acid as reported in the prior art for the condensation takes long time for completion and the reaction suffers both in quality as well as quantity. Surprisingly the use of trifluoro acetic acid alone or in the presence of other solvent enhances the progress of the reaction and the reaction was completed in shorter time which leads high yield.

In another embodiment of the present invention the reaction is carried out at a temperature in the range of 70-200° C. preferably 100-150° C.

In another embodiment of the present invention, the compound of formula (III) was prepared by using 4-hydroxymethyl-5-methylimidazole as a starting material.

In still another embodiment of the present invention, the protecting group represented by R in 4-hydroxymethyl-5-methylimidazole of formula (III) is selected from benzyloxy, t-butyloxy carbonyl (BOC), acetoxy, trityl, phenylmethoxy methyl, t-butyl, benzyl carbomate, acetamide, trifluoro acetamide, 9-fluorenylmethyl carbamate, and methoxymethyl, preferably t-butyloxy carbonyl (BOC). The compound for formula (III) is prepared by treating 4-hydroxymethyl-5-methylimidazole with corresponding reagent for the protecting group as per conventional method. The said reaction is preferably carried out in presence of base such as sodium carbonate, potassium carbonate, sodium hydroxide, potassium hydroxide, sodium bicarbonate and potassium bicarbonate preferably sodium carbonate and in presence of solvent such as alcohol, nitrile, halogenated hydrocarbon preferably acetonitrile.

In still another embodiment of the present invention, the acid used for purification in step b) is selected from organic acid such as acetic acid, formic acid, maleic acid, fumaric acid, p-tolylsulfonic acid, methanesulfonic acid, benzenesulfonic acid and the like.

In still another embodiment of the present invention, the solvent used for purification of step b) is selected from alcohol such as methanol, ethanol, isopropanol, butanol, ketone such as acetone, methyl ethyl ketone, methyl isobutylketone, halogenated hydrocarbon such as dichloromethane, chloroform and water or mixtures thereof preferably acetone.

In still another embodiment of the present invention, the purification of Alosetron of formula (I) removes the impurities such as but not limited to compound of formula (IV).

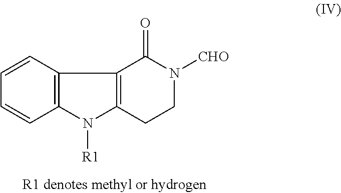

In still another embodiment of the present invention Alosetron of formula (I) prepared according to the present invention having less than 0.1% of above said impurities; preferably less than 0.05%. Accordingly the present invention provides Alosetron having less than 0.1% of compound of formula (IV)

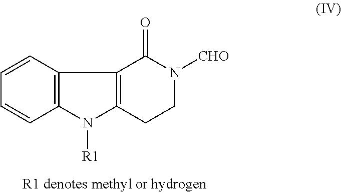

In still another embodiment of the present invention, the Alosetron prepared from the present process having purity greater than 99.3% preferably greater than 99.7%.

In still another embodiment of the present invention, the Alosetron HCl is prepared by treating Alosetron with hydrochloric acid in presence of solvent is selected from ethyl acetate, butyl acetate, acetone methanol, ethanol, isopropanol and butanol or in water or mixture there of. Either anhydrous HCl (like HCl gas or HCl in solvent) or con. hydrochloric acid can be used for the reaction.

Alosetron hydrochloride prepared by present invention is micronized to any size. Preferably 90% of particles having particle size not more than about 75 micron, more preferably 90% of particles having particle size not more than about 25 micron.

The starting material used in the present invention is prepared by utilizing the process available in the literature.

The present invention is provided by the examples below, which are provided by way of illustration only and should not be considered to limit the scope of the invention.

### EXAMPLE 1

### Process for the Preparation of Alosetron

To a mixture of acetic acid and dimethylformamide, 3N-BOC-(-hydroxymethyl-5-methyl imidazole (95.4 g), 2,3,4,5-tetrahydro-5-methyl-1H-pyrido[4,3-b]indol-1-one (50 g), trifluoroacetic acid were added and heated to 100-115° C. After completion of the reaction, the reaction mass was cooled to room temperature. To the reaction mass, carbon was added, stirred and filtered though hyflo bed. The bed was washed with dimethylformamide. The filtrate was distilled under vacuum. To the residue, water was added and washed the reaction mass with toluene followed by isopropyl ether. The pH of the reaction mass was adjusted to 6.8-7 using potassium carbonate solution, stirred, cooled and the obtained solid was dried.

### EXAMPLE 2

### Process for the Preparation of Alosetron

To trifluoroacetic acid, 3N-BOC-4-hydroxymethyl-5-methylimidazole (95.4 g), dimethylformamide (480 mL) and 2,3,4,5-tetrahydro-5-methyl-1H-pyrido[4,3-b]indol-1-one (58 g) were added and heated to 100-115° C. After completion of the reaction, the reaction mass was cooled to room temperature. To the reaction mass, carbon was added and filtered though hyflo bed. The bed was washed with dimethylformamide and the filtrate was distilled under vacuum. To the residue, water was added and washed the aqueous layer with toluene followed by isopropyl ether. The pH of the reaction mass was adjusted to 6.8-7 using potassium carbonate solution, cooled and the obtained solid was dried.

TABLE 1

| Solvent System | Reaction time | Yield |
|---|---|---|
| TFA & DMF | 6-7 hours | 65% |
| acetic acid alone | 20 hours | 17% |
| acetic acid & DMF | No reaction | |

The above table clearly indicates that the use trifluoroacetic acid (TFA) enhances the reaction progress and also increases the yield of the product.

### EXAMPLE 3

### Purification of Alosetron

To acetic acid, crude Alosetron containing >0.2% of compound of formula (IV) was added and heated to 60-65° C., the reaction mass maintained at same temperature and cooled to 40-45° C. To the reaction mass acetone was added and refluxed. The reaction mass was cooled to 0-5° C., the solid obtained was filtered, washed with acetone (slurry) and dried to yield pure Alosetron having less than 0.02% of Impurity of formula (IV) by HPLC. Yield: 53-50 g

### Reference Example 1

### Preparation of Alosetron Hydrochloride

To a methanol (50 mL), Alosetron (10 g) and of IPA.HCl (8.5 mL) were added and heated to 40-45° C. The reaction mass was cooled, stirred and filtered and washed with methanol. The reaction mass was dissolved in methanol, treated with carbon, filtered and washed with methanol. The reaction mass was distilled and isopropyl ether was added to the residue and stirred at room temperature. The reaction mass was cooled, stirred. The solid obtained was filtered and washed with chilled methanol and dried.

### Yield: 7.8 g

### Reference Example-2

### Process for the preparation of 3N-BOC-(4-hydroxymethyl)-5-methylimidazole

4-Hydroxymethyl-5-methylimidazole (100 g) was dissolved in water, to the solution was added sodium carbonate (107 g) and stirred. To the reaction mass acetonitrile (400 mL) was added and cooled to 10-15° C. followed by addition of solution of DIBOC (di-tert-butyl dicarbonate) in acetonitrile. After completion of the reaction, water was added to the reaction mass and filtered. The filtrate was washed with 1:1 acetonitrile and water and than washed with hexane. The mass was extracted with toluene and the organic layer was washed with water followed by brine. The organic layer was distilled under vacuum to get oily mass of the title compound.

We claim:

1. An improved process for the preparation of Alosetron of formula (I) and its pharmaceutically acceptable salts which comprises the steps of: 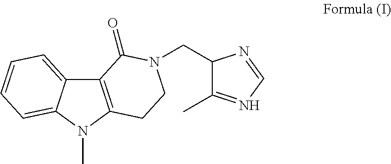
a) condensing 2,3,4,5-tetrahydro-5-methyl-1H-pyrido[4,3-b]indol-1-one of formula (II) with 4-hydroxymethyl-5-methylimidazole of formula (III) in the presence of trifluoroacetic acid and in the presence or absence of solvent to obtain Alosetron of formula (I); and
b) optionally purifying the compound of formula (I) using an organic acid.

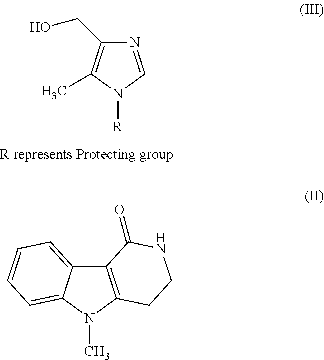

2. The process as claimed in claim 1, wherein the protecting group represented by R is selected from the group consisting of benzyloxy, t-butyloxy carbonyl (BOC), acetoxy, trityl, phenylmethoxy methyl, t-butyl, benzyl carbomate, acetamide, trifluoro acetamide, 9-fluorenylmethyl carbamate and methoxymethyl.

3. The process according to the claim 1, wherein the solvent used in the condensation is selected from dimethylacetamide, dimethylformamide, dimethylsulphoxide, sulfolane, xylene, methanol, ethanol, isopropanol, butanol, acetic acid, formic acid or mixture thereof.

4. The process according to the claim 1, wherein the organic acid used in step b) is selected from acetic acid, formic acid, maleic acid, fumaric acid, p-tolyl sulfonic acid, methane sulfonic acid, benzene sulfonic acid or mixtures thereof.

5. A process for the purification of Alosetron comprises treating Alosetron with an organic acid in presence or absence of solvent.

6. A process as claimed in claim 5, wherein the organic acid is acetic acid.

7. The process according to the claim 5, wherein the solvent is selected from methanol, ethanol, isopropanol, butanol, acetone, methyl ethyl ketone, methyl isobutylketone, dichloromethane, chloroform and water or mixtures thereof; preferably acetone.

8. Alosetron having less than 0.1% of compound of formula (IV) [chemical structure]
